(19) United States
(12) Patent Application Publication
(10) Pub. No.: US 2026/0108399 A1
(43) Pub. Date: Apr. 23, 2026

(54) EMERGENCY MEDICAL KIT WITH MODULAR ORGANIZATION SYSTEM

(71) Applicant: Alexander Hauk, Springfield, OR (US)

(72) Inventor: Alexander Hauk, Springfield, OR (US)

(21) Appl. No.: 18/919,722

(22) Filed: Oct. 18, 2024

(51) Int. Cl. A61F 17/00

(52) U.S. Cl. CPC A61F 17/00

(57) ABSTRACT

An emergency medical kit may comprise a main body with multiple compartments. A fabric panel may be attached to the main body. The fabric panel may include horizontal and vertical straps forming a grid pattern. The fabric panel may have attachment points for securing medical supplies. A removable medication pack containing pre-selected medications may be included. A removable wound cleaning pack with treatment supplies may also be provided. The kit may further include items like a blood pressure cuff, stethoscope, tourniquet, trauma shears, and pulse oximeter. The fabric panel may have a mesh pocket with a zipper. The main body may open in a T-shaped layout. Elastic bands may secure additional supplies. The grid pattern may comprise a MOLLE system. A medical bag component with a mesh pocket and logo may attach to the main body.

### FIELD OF THE INVENTION

The present disclosure relates generally to medical kits, and more particularly to an emergency medical kit with a modular organization system for efficient storage and access to medical supplies.

### BACKGROUND

Emergency medical situations often require quick access to a variety of medical supplies and equipment. Conventional medical kits may lack efficient organization systems, making it difficult for users to quickly locate and retrieve necessary items in high-pressure situations. Additionally, many existing kits lack modularity and customization options to suit different emergency scenarios or user preferences. There exists a need for an improved emergency medical kit that addresses these limitations and provides enhanced organization, accessibility, and adaptability for various emergency medical situations.

### SUMMARY

The present disclosure is directed to an emergency medical kit with a modular organization system. The kit may include a main body with multiple compartments and attachment points for organizing and securing various medical supplies and equipment. The kit may further include removable and customizable modules for storing specific types of supplies or medications. In one aspect, the emergency medical kit may comprise a fabric panel with horizontal and vertical straps forming a grid pattern for attaching various components and accessories. The kit may also include multiple zippered compartments, mesh pockets, and removable pouches for organizing different types of medical supplies.

In another aspect, the emergency medical kit may feature a modular medication pack and a wound cleaning pack, each containing pre-selected supplies for specific medical needs. These packs may be easily removable and replaceable within the main kit structure. The emergency medical kit may also include various medical devices and tools, such as a blood pressure cuff, stethoscope, tourniquet, and trauma shears, securely organized within the kit for quick access during emergencies. These and other features, aspects, and advantages of the present disclosure will become better understood with reference to the following description and appended claims.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings indicates similar or identical items. The accompanying drawings, which are incorporated in and constitute a part of this specification, illustrate embodiments of the disclosure and, together with a general description of the disclosure given above, and the detailed description given below, serve to explain the principles of the disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. The present disclosure, in accordance with one or more various embodiments, is described in detail with reference to the following figures. The drawings are provided for purposes of illustration only and merely depict typical or example embodiments of the disclosure. These drawings are provided to facilitate the reader's understanding of the disclosure and shall not be considered limiting of the breadth, scope, or applicability of the disclosure.

FIG. 1 is a diagram illustrating the contents and organization of an exemplary emergency medical kit according to one embodiment of the present disclosure.

FIG. 2 is a perspective view of an exemplary emergency medical kit in an open configuration according to one embodiment of the present disclosure.

FIG. 3 is a front view of a fabric panel with organizational features according to one embodiment of the present disclosure.

FIGS. 4A-4F are detailed views of various components and features of the emergency medical kit according to embodiments of the present disclosure.

FIGS. 5A-5B are perspective views of a medical bag component of the emergency medical kit according to embodiments of the present disclosure.

### DETAILED DESCRIPTION

The present disclosure provides an emergency medical kit with a modular organization system. It should be appreciated that this description is for illustrative purposes only and is not intended to limit the scope of the present disclosure. In addition, various features are described herein in a certain order or arrangement, but such features may be combined, separated, or rearranged without departing from the spirit and scope of the disclosure. In other instances, well-known features have not been described in detail to avoid unnecessarily complicating the description.

Throughout this specification and the claims, the terms “comprise,” “comprising,” “include,” “including,” and the like are to be understood to imply the inclusion of stated elements but not the exclusion of any other elements. The term “exemplary” is used in the sense of “example” rather than “ideal” or “model.” As used herein, the terms “about” or “approximately” apply to all numeric values, whether or not explicitly indicated. These terms generally refer to a range of numbers that one of skill in the art would consider equivalent to the recited values (i.e., having the same function or result).

The present disclosure includes many aspects and features. Moreover, while many aspects and features relate to, and are described in, the context of systems and methods of fraud identification, embodiments of the present disclosure are not limited to use only in this context. The present disclosure can be understood more readily by reference to the following detailed description of the disclosure and the examples included therein.

Before the present articles, systems, apparatuses, and/or methods are disclosed and described, it is to be understood that they are not limited to specific methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

### A. Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise. As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

It is understood that the apparatuses and systems disclosed herein have certain functions. Disclosed herein are certain structural requirements for performing the disclosed functions, and it is understood that there are a variety of structures that can perform the same function that are related to the disclosed structures, and that these structures will typically achieve the same result. The following description of various embodiments is merely exemplary in nature and is in no way intended to limit the disclosure, its application, or uses.

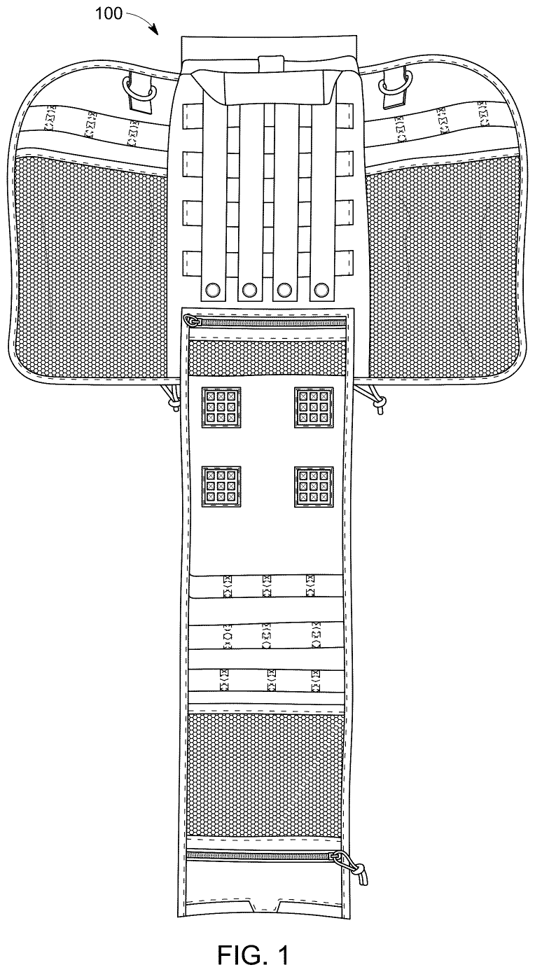

Referring now to FIG. 1, an exemplary layout of the contents of an emergency medical kit 100 is illustrated. The kit 100 may include various sections, each containing specific medical supplies and equipment. In the illustrated embodiment, the kit 100 may include a carabiner, CPR mask, and whistle in section 1; a blood pressure cuff and stethoscope in section 2; a tourniquet and shoulder strap in section 3; writing instruments such as a Sharpie pen and a Bic pen in section 4; trauma shears with an O2 key in section 5; a metal flashlight in section 6; various instruction guides and a pulse oximeter in section 7; and a JumpMedic patch in section 8. The emergency medical kit 100 may also include a medication pack containing various medications such as antihistamines, pain relievers, antidiarrheal medication, and oral relief medication. Additionally, a wound cleaning pack may be included, containing items such as bandages, antibiotic ointment, alcohol prep pads, and wound closure strips.

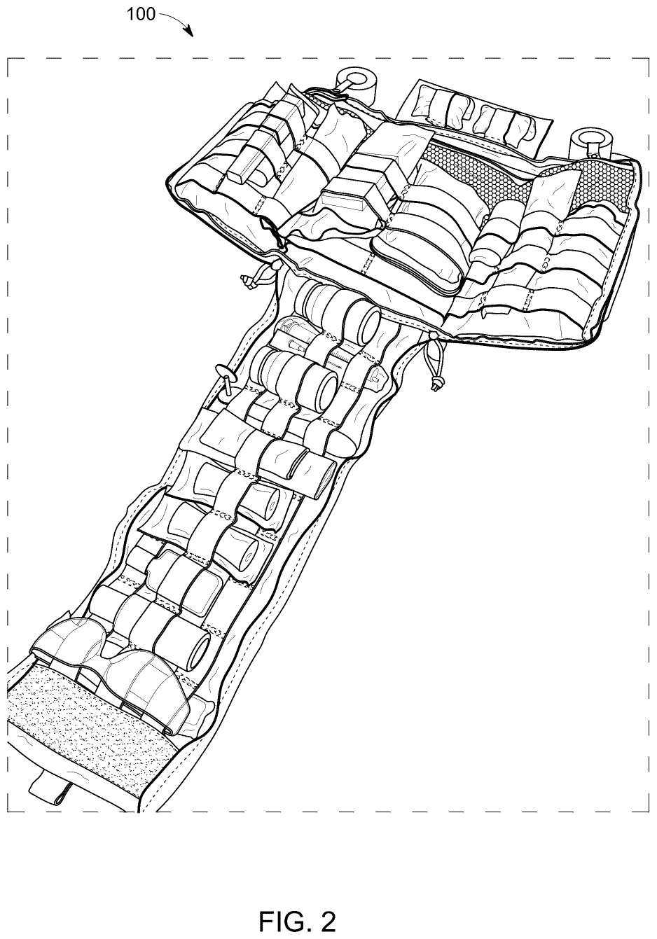

FIG. 2 illustrates an exemplary emergency medical kit 100 in an open configuration 200. The emergency medical kit 100 in an open configuration 200 may have a T-shaped layout when fully opened, providing easy access to multiple compartments. The emergency medical kit 100 in an open configuration 200 may include red borders and black pockets and straps for organizing various medical supplies. Elastic bands may be used to secure items such as rolls of medical tape and smaller supplies.

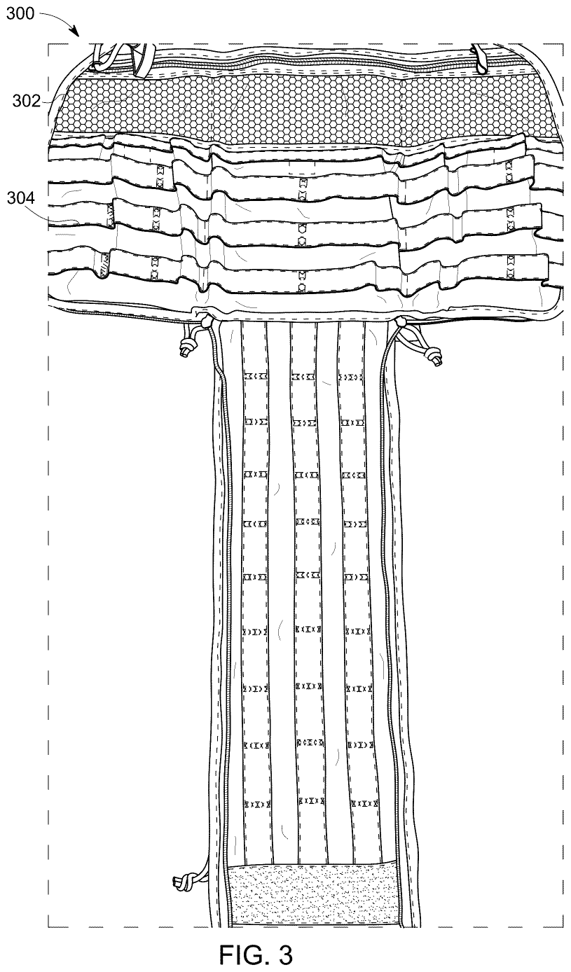

Referring to FIG. 3, a fabric panel 300 of the emergency medical kit 100 is shown. The panel 300 may feature red and black horizontal stripes and may include a mesh pocket 302 with a zipper at the top. Below the mesh pocket 302, multiple horizontal loops or straps 304 may be sewn across the width of the panel 300. This configuration may allow for customizable organization and attachment of various medical supplies and equipment.

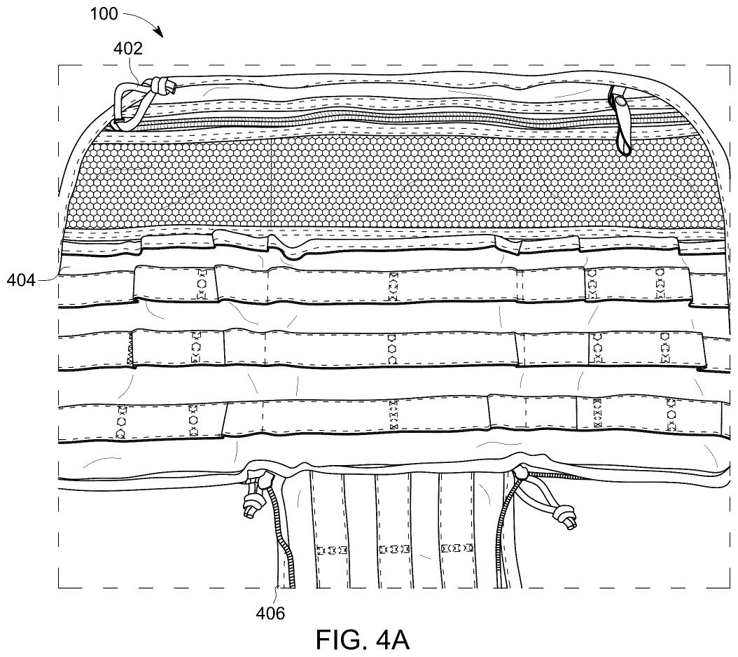

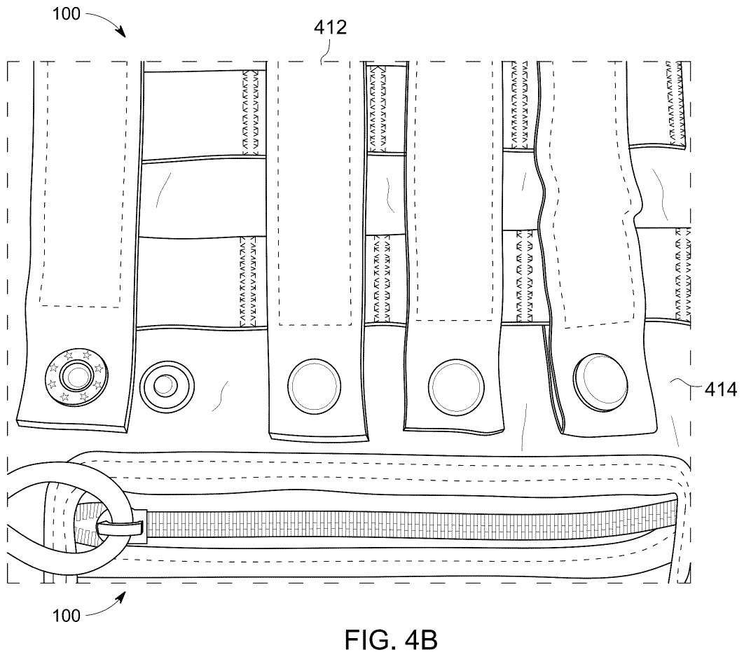

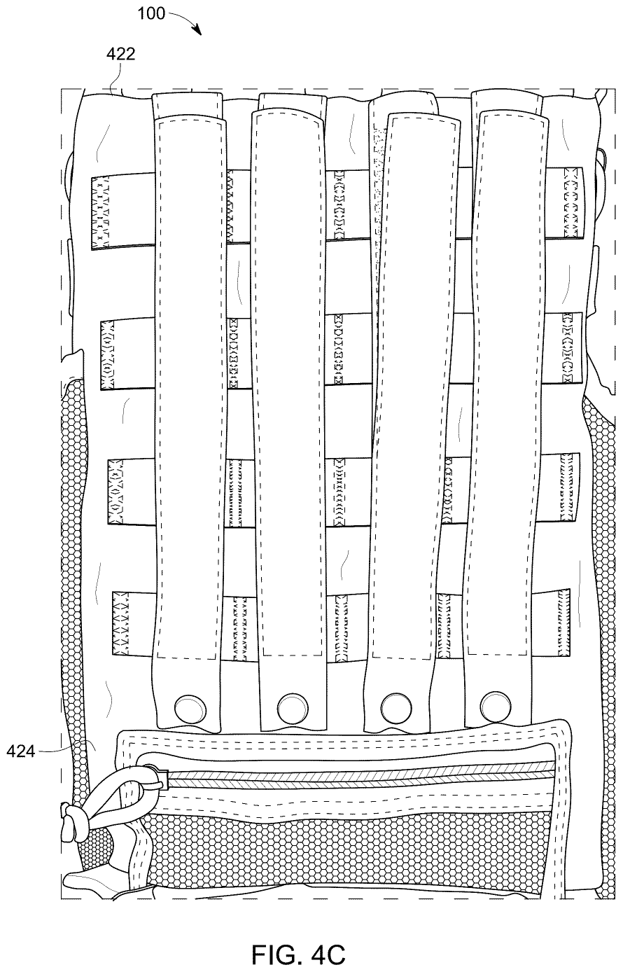

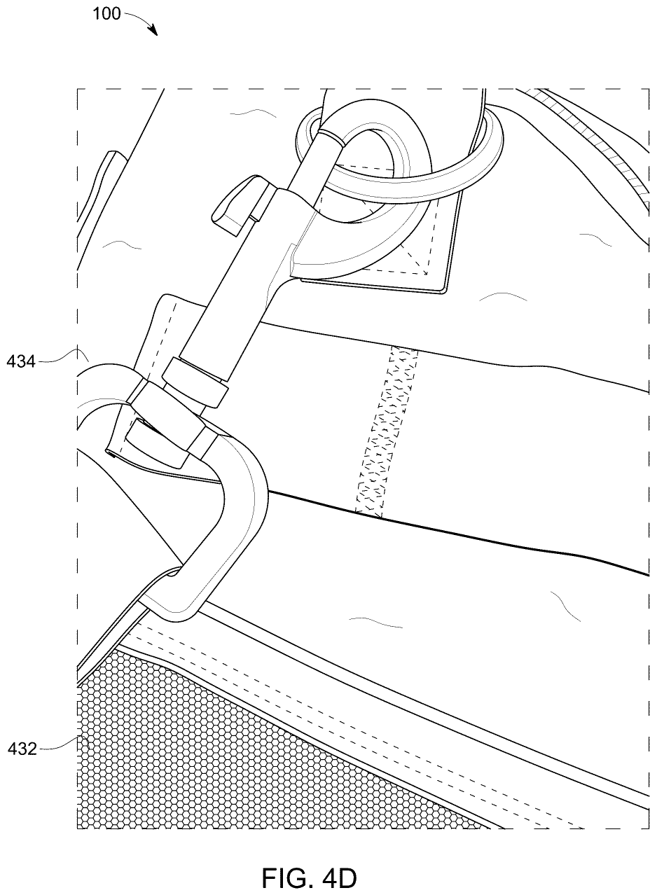

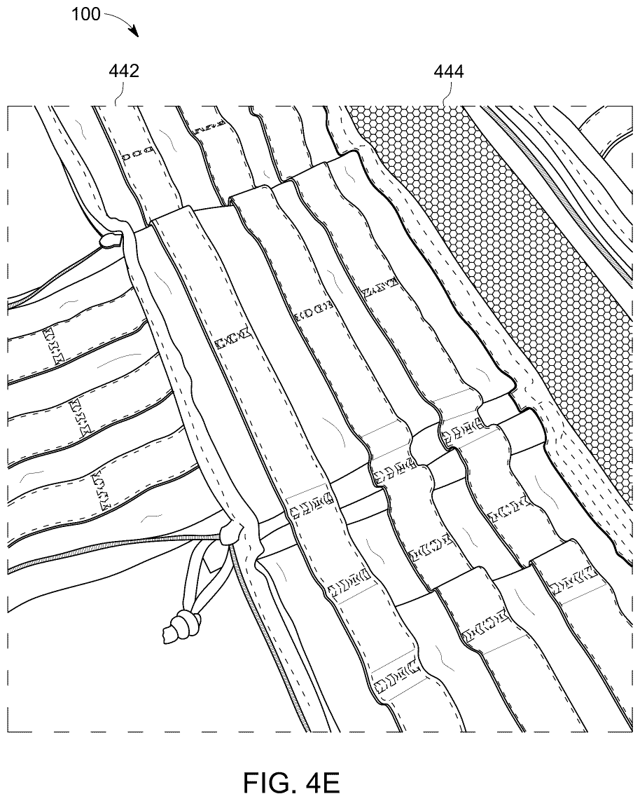

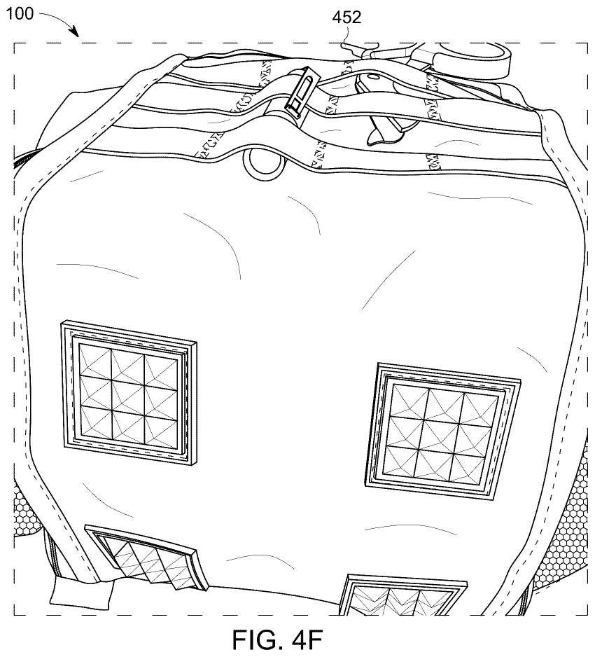

FIGS. 4A-4F illustrate detailed views of various components and features of the emergency medical kit. FIG. 4A shows a view of the emergency medical kit 100 with a zippered compartment 402 at the top, followed by a netted red pocket 404. Below the pocket 404, multiple horizontal black straps 406 may be layered over a red fabric base, forming a grid structure for storage or attachment points. FIG. 4B depicts a section of the emergency medical kit 100 with black straps 412 sewn onto it in a grid pattern. The emergency medical kit 100 may include metal snap buttons and plastic snap buttons 414, as well as a black zipper 416 with a circular pull tab. FIG. 4C shows the emergency medical kit 100 with multiple vertical black straps 422 running across it. The straps 422 may be interwoven between horizontal loops, potentially forming a Modular Lightweight Load-carrying Equipment (MOLLE) system. A black zipper pocket 424 with a mesh overlay may be located at the bottom of the panel. FIG. 4D illustrates a close-up view of the emergency medical kit 100 featuring black webbed straps 432. A black metal swivel hook or clip 434 may be attached to the straps 432, providing a means for securing or hanging the kit or its components. FIG. 4E shows the emergency medical kit 100 with black straps 442 running horizontally across its width. The emergency medical kit 100 may be foldable and may include a zipper 444 with a drawstring on one side. A mesh-like pattern 446 may be visible on the opposite side. FIG. 4F depicts a bottom portion of the emergency medical kit 100. In one or more embodiments, the emergency medical kit 100 may comprise multiple straps 452 with buckles on its top side.

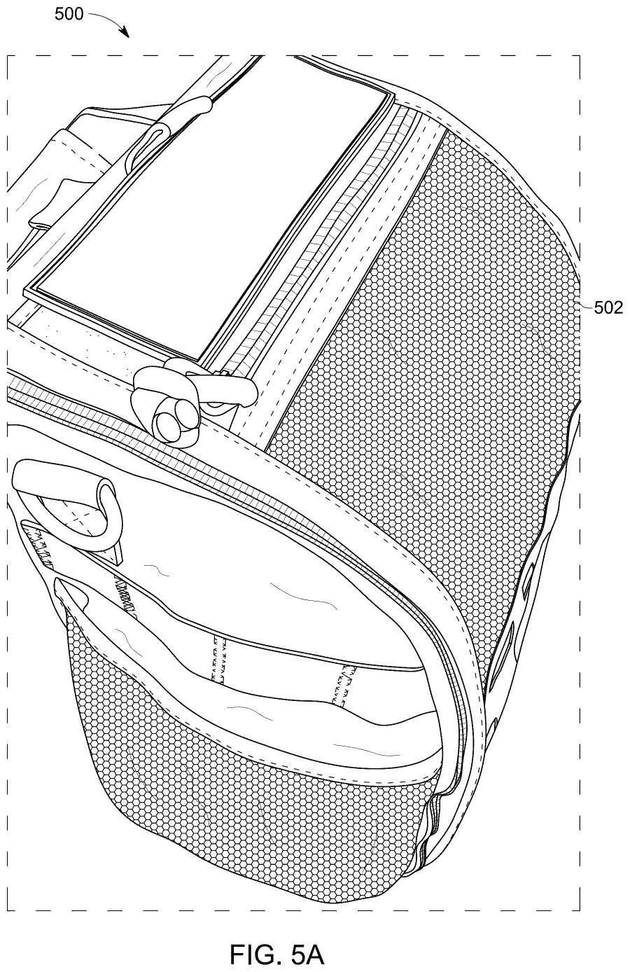

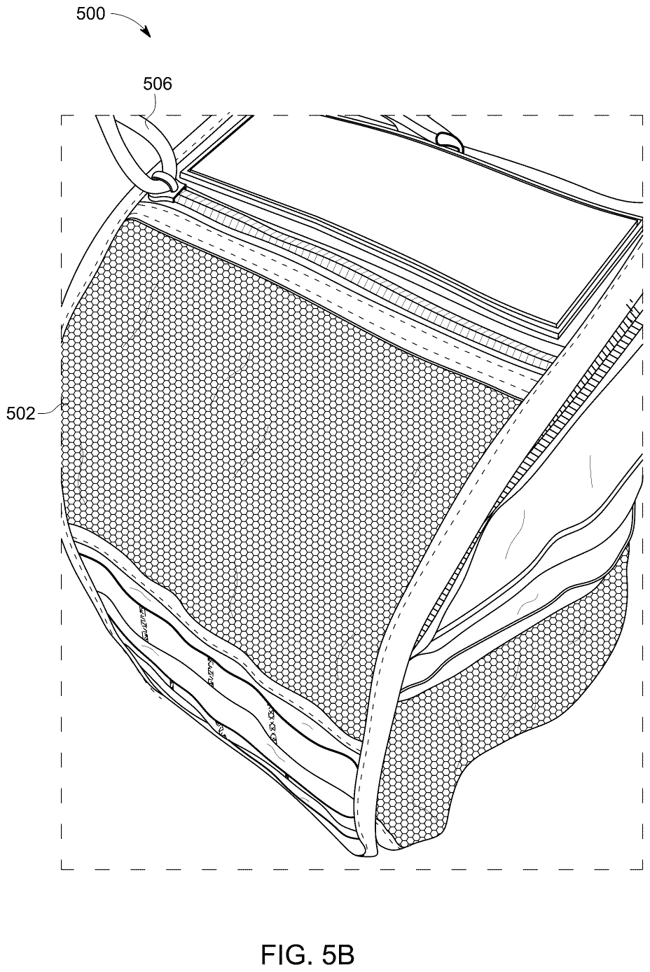

FIGS. 5A and 5B illustrate perspective views of a fully folded and closed embodiment of the emergency medical kit 100 of the emergency medical kit. The folded emergency medical kit 500 may have a red and black color scheme and may include a mesh pocket 502. A logo 504 displaying the text “JumpMedic” in red lettering on a black background may be prominently featured. The folded emergency medical kit 500 may also include a zipper 506 on top and a fabric loop for carrying. In operation, the emergency medical kit may provide an organized and efficient system for storing and accessing various medical supplies and equipment. The modular design may allow users to customize the kit's contents and organization to suit specific needs or preferences. The use of different colored fabrics, straps, and pockets may aid in quickly identifying and locating specific items within the kit. The kit may be designed for use in various emergency medical situations, such as first response scenarios, outdoor adventures, or workplace emergencies. The inclusion of pre-packaged medication and wound cleaning kits may allow for quick access to commonly needed supplies, while the modular organization system may enable easy restocking and customization.

The emergency medical kit may further include a variety of additional components and features to enhance its functionality and versatility. For example, the kit may incorporate a modular design that allows users to customize the contents based on specific needs or environments. The fabric panel with the grid pattern of horizontal and vertical straps may utilize a standardized attachment system, such as MOLLE (Modular Lightweight Load-carrying Equipment). This system may enable users to easily add, remove, or rearrange pouches, pockets, and other accessories as needed. The MOLLE-compatible straps may be reinforced and stitched securely to the fabric panel to ensure durability and reliable attachment of components.

In addition to the mesh pocket with a zipper shown in FIG. 3, the kit may include multiple types of storage options integrated into the fabric panels. These may include hook-and-loop fastener patches for attaching removable pouches, elastic loops for securing cylindrical items like medication vials or pens, and transparent plastic windows to allow visibility of contents without opening compartments.

The main body of the kit, as depicted in FIG. 2, may be constructed from durable, water-resistant materials to protect the contents from environmental factors. The T-shaped layout when fully opened may be designed to provide maximum accessibility to all compartments while maintaining a compact folded size for storage and transport. The kit may also feature reinforced stitching at stress points and heavy-duty zippers to withstand frequent use in challenging conditions.

To enhance organization and rapid access to supplies, the kit may employ a color-coding system. Different sections or modules may be assigned specific colors or color combinations to indicate the types of supplies contained within. For example, red may be used for bleeding control supplies, blue for airway management tools, and green for medication modules. This color-coding may extend to the fabric panels, straps, and even individual pouches or containers within the kit.

The removable medication pack and wound cleaning pack may be designed with clear labeling and compartmentalization to facilitate quick identification and access to specific items. These packs may also incorporate tamper-evident seals to ensure the integrity of the contents and allow for easy identification of used or expired supplies that need replacement.

The medical bag component shown in FIGS. 5A and 5B may be designed to be either permanently attached to the main kit or removable for use as a standalone unit. This bag may feature additional organizational elements such as internal dividers, elasticized instrument holders, and detachable smaller pouches for specific procedures or supply sets.

To address various emergency scenarios, the kit may include specialized modules or inserts designed for specific medical situations. These may include a burn treatment module, a dental emergency kit, or a hypothermia management pack. Each specialized module may be color-coded and labeled for quick identification and may be easily integrated into the main kit using the MOLLE attachment system. The emergency medical kit may also incorporate technological features to enhance its capabilities. For example, it may include a built-in rechargeable battery pack with USB ports to power electronic medical devices or charge communication equipment. Additionally, the kit may feature RFID (Radio-Frequency Identification) tags on individual components to facilitate inventory tracking and automatic reordering of depleted supplies. To improve usability in low-light conditions, the kit may incorporate reflective strips or patches on the exterior. These reflective elements may aid in locating the kit in darkness and may also serve to increase visibility when the kit is being used in roadside emergencies.

The fabric loop shown in FIG. 5A may be part of a larger carrying system that includes adjustable shoulder straps and a waist belt. This system may allow the kit to be worn as a backpack, sling bag, or attached to other equipment, providing versatility in how the kit can be transported and accessed in different situations. Overall, the emergency medical kit may be designed with a focus on modularity, durability, and efficient organization to provide medical responders with a versatile and reliable tool for addressing a wide range of emergency situations. The combination of standardized attachment systems, color-coding, and specialized modules may allow for customization and rapid access to critical supplies when time is of the essence.

The emergency medical kit may be utilized in various emergency situations to provide immediate medical assistance. For example, in a car accident scenario, the kit may be quickly deployed to address multiple injuries. The blood pressure cuff and stethoscope may be used to assess vital signs of injured individuals. The tourniquet may be applied to control severe bleeding from limb injuries. The CPR mask may be utilized if cardiopulmonary resuscitation is required. In a hiking emergency, the whistle may be used to signal for help. The metal flashlight may aid in navigation and examination of injuries in low light conditions. The wound cleaning pack may be employed to clean and dress cuts or abrasions sustained on the trail. Pain relief medications from the medication pack may be administered as needed. During a workplace incident, such as a fall or machinery accident, the trauma shears may be used to quickly remove clothing to access injuries. The pulse oximeter may monitor oxygen levels of an injured worker. The JumpMedic patch may be applied to control bleeding. The first aid guide may provide step-by-step instructions for treating specific workplace injuries.

In a natural disaster scenario, the kit's modular design may allow for customization based on anticipated needs. The shoulder strap may facilitate hands-free carrying of the kit through debris-filled areas. The antiemetic medication may help individuals experiencing nausea due to stress or contaminated water. The oral relief medication may soothe irritation from exposure to smoke or dust. For a sports-related injury, such as a sprained ankle, the kit's ice pack may be activated to reduce swelling. The elastic bandages may be used to wrap and support the injured joint. Pain relievers from the medication pack may be administered to manage discomfort.

In a school setting, the kit may address various situations. The antihistamine may be used for allergic reactions. The glucometer may monitor blood sugar levels for diabetic students. The splinter remover may extract foreign objects from minor playground injuries. During a home emergency, such as a kitchen accident, the burn gel may be applied to minor burns. The tweezers may remove small debris from wounds. The antibiotic ointment may be used to prevent infection in cuts. In all these scenarios, the kit's organized layout and color-coded sections may allow for quick identification and access to needed supplies. The modular design may enable customization for specific environments or anticipated emergencies. The durable construction may withstand use in challenging conditions, ensuring reliability when needed most.

It should be understood that the embodiments described herein are exemplary and that a person skilled in the art may make many variations and modifications without departing from the spirit and scope of the disclosure. All such variations and modifications are intended to be included within the scope of the disclosure as defined in the appended claims.

While illustrative embodiments of the invention have been shown and described, variations and alternative embodiments may occur to those skilled in the art. Such variations and alternative embodiments may be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom.

Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112(f), unless the element is expressly recited using the exact phrase “means for . . . ” and no method or process step herein is to be construed under the provisions of 35 U.S.C. section 112(f) unless the step, or steps, are expressly recited using the exact phrase “step(s) for . . . ”.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications can be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior present disclosure. Further, the dates of publication provided herein can be different from the actual publication dates, which can require independent confirmation.

The patentable scope of the present disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

Insofar as the description above and the accompanying drawing disclose any additional subject matter that is not within the scope of the claims below, the disclosures are not dedicated to the public and the right to file one or more applications to claims such additional disclosures is reserved.

The foregoing descriptions of specific embodiments of the present invention have been presented for purposes of illustration and description. They are not intended to be exhaustive or to limit the present invention to the precise forms disclosed, and modifications and variations are possible in view of the above teaching. The exemplary embodiment was chosen and described to best explain the principles of the present invention and its practical application, to thereby enable others skilled in the art to best utilize the present invention and its embodiments with modifications as suited to the use contemplated.

It is therefore submitted that the present invention has been shown and described in the most practical and exemplary embodiments. It should be recognized that departures may be made which fall within the scope of the invention. With respect to the description provided herein, it is submitted that the optimal features of the invention include variations in size, materials, shape, form, function and manner of operation, assembly, and use. All structures, functions, and relationships equivalent or essentially equivalent to those disclosed are intended to be encompassed by the present invention.

It should be understood that the above-described embodiments are illustrative of only a few of the possible specific embodiments which can represent applications of the principles of the present disclosure. Numerous and varied other arrangements can be readily devised by those skilled in the art without departing from the spirit and scope of the disclosure.

What is claimed:

1. An emergency medical kit comprising:
a main body having a plurality of compartments;
a fabric panel attached to the main body, the fabric panel comprising a plurality of horizontal and vertical straps forming a grid pattern;
a plurality of attachment points on the fabric panel for securing medical supplies;
a removable medication pack containing a pre-selected set of medications; and
a removable wound cleaning pack containing wound treatment supplies.

2. The emergency medical kit of claim 1, further comprising:
a blood pressure cuff;
a stethoscope;
a tourniquet;
trauma shears; and
a pulse oximeter.

3. The emergency medical kit of claim 1, wherein the fabric panel comprises a mesh pocket with a zipper.

4. The emergency medical kit of claim 1, wherein the main body is configured to open in a T-shaped layout.

5. The emergency medical kit of claim 1, further comprising a plurality of elastic bands for securing medical supplies.

6. The emergency medical kit of claim 1, wherein the fabric panel comprises a combination of red and black materials.

7. The emergency medical kit of claim 1, further comprising a medical bag component having a mesh pocket and a logo.

8. The emergency medical kit of claim 7, wherein the medical bag component comprises a zipper and a fabric loop for carrying.

9. The emergency medical kit of claim 1, wherein the grid pattern formed by the horizontal and vertical straps comprises a Modular Lightweight Load-carrying Equipment (MOLLE) system.

10. The emergency medical kit of claim 1, further comprising a plurality of snap buttons for securing components of the kit.

11. A method of organizing medical supplies in an emergency kit, the method comprising:
providing a main body having a plurality of compartments;
attaching a fabric panel to the main body, the fabric panel comprising a plurality of horizontal and vertical straps forming a grid pattern;
securing medical supplies to the fabric panel using a plurality of attachment points;
inserting a removable medication pack containing a pre-selected set of medications into one of the plurality of compartments; and
inserting a removable wound cleaning pack containing wound treatment supplies into another of the plurality of compartments.

12. The method of claim 11, further comprising arranging the main body in a T-shaped layout when opened.

13. The method of claim 11, further comprising securing additional medical supplies using elastic bands attached to the main body.

14. The method of claim 11, further comprising attaching a medical bag component to the main body, the medical bag component having a mesh pocket and a logo.

15. The method of claim 11, wherein the grid pattern formed by the horizontal and vertical straps comprises a Modular Lightweight Load-carrying Equipment (MOLLE) system.

16. A modular emergency medical kit system comprising:
a main body having a plurality of compartments;
a fabric panel attached to the main body, the fabric panel comprising a plurality of horizontal and vertical straps forming a grid pattern;
a plurality of removable and interchangeable modules, each module containing specific medical supplies or equipment;
a plurality of attachment points on the fabric panel for securing the removable and interchangeable modules; and
a medical bag component attachable to the main body, the medical bag component having a mesh pocket and a logo.

17. The modular emergency medical kit system of claim 16, wherein the removable and interchangeable modules comprise:
a medication module containing a pre-selected set of medications; and
a wound cleaning module containing wound treatment supplies.

18. The modular emergency medical kit system of claim 16, wherein the grid pattern formed by the horizontal and vertical straps comprises a Modular Lightweight Load-carrying Equipment (MOLLE) system.

19. The modular emergency medical kit system of claim 16, wherein the main body is configured to open in a T-shaped layout.

20. The modular emergency medical kit system of claim 16, further comprising a plurality of snap buttons for securing the removable and interchangeable modules to the main body.
